(19) United States
(12) Patent Application Publication
(10) Pub. No.: US 2017/0080008 A1
(43) Pub. Date: Mar. 23, 2017

(54) STABLE PHARMACEUTICAL COMPOSITIONS OF SOFOSBUVIR

(71) Applicant: Galenicum Health S.L., Barcelona (ES)

(72) Inventor: Sergio Hidalgo ARROYO, Barcelona (ES)

(21) Appl. No.: 15/123,006

(22) PCT Filed: Mar. 5, 2015
(86) PCT No.: PCT/EP2015/054566

§ 371 Date: Sep. 1, 2016

(30) Foreign Application Priority Data
Mar. 5, 2014 (EP) 14157798.1

(51) Int. Cl. A61K 31/7072; A61K 9/20; A61K 31/439; A61K 9/00

(52) U.S. Cl. CPC A61K 31/7072; A61K 9/0053; A61K 9/2009; A61K 9/2027; A61K 9/2013; A61K 9/2018; A61K 9/2054; A61K 9/2095; A61K 31/439

(57) ABSTRACT

The present invention relates to stable pharmaceutical compositions of sofosbuvir or a pharmaceutically acceptable salt thereof comprising at least one pharmaceutically acceptable excipient, in the form of immediate release tablets, to a process for the manufacture of said stable pharmaceutical compositions and to uniform pharmaceutical batches of said immediate release tablets.

The present invention relates to stable pharmaceutical compositions of sofosbuvir or a pharmaceutically acceptable salt thereof comprising at least one pharmaceutically acceptable excipient, in the form of immediate release tablets, to a process for the manufacture of said stable pharmaceutical compositions and to uniform pharmaceutical batches of said immediate release tablets.

### STATE OF THE ART

Hepatitis C virus (HCV) infection is a major health problem that leads to chronic liver disease, such as cirrhosis and hepatocellular carcinoma, in a substantial number of infected individuals, estimated to be 3% of the world's population. According to the World Health Organization, there are more than 200 million infected individuals worldwide, with at least 3 to 4 million people being infected each year. Once infected, about 20% of people clear the virus, but the rest can harbor HCV the rest of their lives. About 10 to 20% of chronically infected individuals eventually develop liver-destroying cirrhosis or cancer. The viral disease is transmitted parenterally by contaminated blood and blood products, contaminated needles, or sexually and vertically from infected mothers or carrier mothers to their offspring.

Current treatments for HCV infection, which are restricted to immunotherapy with recombinant interferon-α alone or in combination with the nucleoside analog ribavirin, are of limited clinical benefit. In particular, interferon-based treatment is not suitable for many patients with hepatitis C virus (HCV) infection because of contraindications such as psychiatric illness, and a high burden of adverse events. Moreover, there is no established vaccine for HCV. Consequently, there is an urgent need for improved therapeutic agents that effectively combat chronic HCV infection.

Sofosbuvir (also called GS-7977 and formerly called PSI-7977) is a nucleotide analog prodrug currently in Phase 2/Phase 3 trials for treatment of chronic HCV infection. Several Phase 2 clinical trials have been conducted to evaluate the efficacy, safety and tolerability of sofosbuvir 400 mg administered for 8 or 12 weeks with or without ribavirin and optionally peginterferon in subjects with genotype 1, genotype 2 or genotype 3 HCV. The results of these trials, along with the results of in vitro studies, revealed several potential and hereto unknown advantages of HCV treatment regimens utilizing sofosbuvir in combination with ribavirin. These results provide a basis for the disclosed and claimed method and composition for treating HCV infection.

Sofosbuvir, isopropyl (2S)-2-[[[(2R,3R,4R,5R)-5-(2,4-dioxopyrimidin-1-yl)-4-fluoro-3-hydroxy-4-methyl-tetrahydrofuran-2-yl]methoxy-phenoxy-phosphoryl]amino]propanoate is described in U.S. Pat. No. 7,964,580. (See also US 2010/0016251, US 2010/0298257, US 201 1/0251 152 and US 2012/0107278). Sofosbuvir has the following structure:

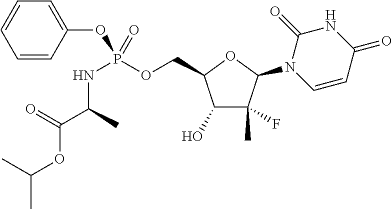 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

There is a need for improved treatment regimens that are more effective, safe, tolerable and shorter in duration, which are associated with reduced rates of viral breakthrough and/or viral resistance. In particular, there is a need for interferon-free treatment regimens that are effective for treating chronic hepatitis C (“CHC”) but result in reduced side-effects compared to treatment regimens involving interferon or peginterferon. There is also a need for interferon-free treatment regimens for patients suffering from CHC infection who are interferon-ineligible or interferon-intolerant.

Furthermore, there is still a need to provide an improved dosage form which would comprise a stable pharmaceutical composition of sofosbuvir in a smaller tablet.

A suitable technical solution of how to adapt the size and composition of sofosbuvir tablets to fulfill the above requirement has not been addressed in the prior art and thus there is a need for such a solution. An improvement to simplify treatment regimens for hepatitis C would enhance patient compliance by providing a simplified dosage form being more concentrate.

HCV infection is associated with progressive liver pathology, including cirrhosis and hepatocellular carcinoma. Chronic hepatitis C may be treated with peginterferon-alpha in combination with ribavirin. Substantial limitations to efficacy and tolerability remain as many users suffer from side effects, and viral elimination from the body is often inadequate. Therefore, there is a need for new drugs and new drug combinations to treat HCV infection, in particular chronic hepatitis C.

Ledipasvir, also named GS-5885, is an experimental drug for the treatment of hepatitis C. It is currently in Phase III clinical trials. It is being studied in combination with other direct-acting antiviral agents that interfere with HCV replication. Ledipasvir is an inhibitor of the hepatitis C virus HCV NS5A protein. Ledipasvir is being tested in interferon-free regimens with other direct-acting antiviral agents for hepatitis C.

Ledipasvir, has chemical name carbamic acid N-[(1S)-1-[[(6S)-6-[5-[9,9-difluoro-7-[2-[(1R,3S,4S)-2-[(2S)-2-[(methoxycarbonyl)amino]-3-methyl-1-oxobutyl]-2-azabicyclo[2.2.1]hept-3-yl]-1H-benzimidazol-6-yl]-9H-fluoren-2-yl]-1H-imidazol-2-yl]-5-azaspiro[2.4]hept-5-yl]carbonyl]-2-methylpropyl]-, methyl ester. Ledipasvir has the following structure:

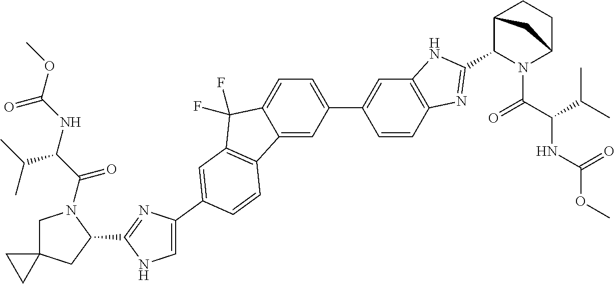 [SMILES: COC(=O)N[C@H](C(=O)N1CC2(CC2)C[C@H]1c1nc(-c2ccc3c(c2)C(F)(F)c2ccc(-c4ccc5nc([C@@H]6[C@@H]7CC[C@@H](C7)N6C(=O)[C@@H](NC(=O)OC)C(C)C)[nH]c5c4)cc2-3)c[nH]1)C(C)C]

There is still a need to provide a dosage form which would comprise a combination of sofosbuvir and ledipasvir for the treatment of chronic hepatitis C genotype 1 infection in adults. Said pharmaceutical composition being a stable and having a fast onset.

The above issues have been addressed in the present invention and specific embodiments thereof.

### SUMMARY OF THE INVENTION

The present invention provides stable pharmaceutical compositions of sofosbuvir or a pharmaceutically acceptable salt thereof in the form of immediate release tablets. The stable immediate release tablets as disclosed herein can be stored at temperatures higher than 25° C. and are stable even when packaged in aluminium/PVC.

Furthermore, the present invention provides a pharmaceutical composition in the form of an immediate release tablet comprising sofosbuvir, which has a smaller size than the tablets of the prior art, whereas maintaining the stability, bioavailability and efficacy of the pharmaceutical composition.

The compositions of the present invention offer the additional benefit of increasing the concentration of sofosbuvir per total weight of the tablet, which permits smaller tablets to be used to deliver the same dosage of the drug.

In particular, such a kind of pharmaceutical composition is advantageous since it would require a lower quantity of excipients. Preferably all this would result in both economic (cheaper cost of formulation, packaging, etc.) and will have an advantage for patients with swallowing difficulties as elderly, enhancing the treatment adherence or compliance for these patients.

In a first aspect, the present invention relates to an oral solid pharmaceutical composition comprising sofosbuvir or a pharmaceutically acceptable salt thereof, wherein said pharmaceutical composition comprises:
a) more than about 35% w/w of sofosbuvir as a free base in respect of the total amount of the pharmaceutical composition; and
b) at least one pharmaceutically acceptable excipient.

In a second aspect, the present invention relates to a granulate comprising sofosbuvir or a pharmaceutically acceptable salt thereof, wherein the granulate comprises from about 25% to about 55% w/w of sofosbuvir as free base in respect of the total amount of the granulate and at least one disintegrant and at least one filler.

In a third aspect, the present invention relates to a pharmaceutical composition comprising the granulate according to the second aspect.

In a fourth aspect, the present invention relates to the amorphous form of sofosbuvir for use in the manufacture of a pharmaceutical composition.

In a fifth aspect, the present invention relates to the amorphous form of sofosbuvir for use in the manufacture of the pharmaceutical composition of the first or third aspect or the granulate of the second aspect.

In a sixth aspect, the present invention relates to a pharmaceutical batch comprising at least 20,000 units of the pharmaceutical composition of the first or third aspect.

In a seventh aspect, the present invention relates to the pharmaceutical composition of the first or third aspect, the granulate of the second aspect, or the pharmaceutical batch of the sixth aspect, for use in the treatment of hepatitis C.

In an eighth aspect, the present invention relates to a drug product comprising at least:
i) a pharmaceutical composition in the form of tablet comprising sofosbuvir or a pharmaceutically acceptable salt thereof, and
ii) a blister pack packaging the pharmaceutical composition (i).

In a ninth aspect, the present invention relates to a process for the manufacture of the pharmaceutical composition of the first or third aspect or the pharmaceutical batch of the sixth aspect, wherein the process comprises the following steps:
(a) providing an intragranular mixture of sofosbuvir or a pharmaceutically acceptable salt thereof and at least one disintegrant and at least one filler;
(b) granulating the mixture by a dry granulation process to produce a granulate;
(c) blending the granulate of step (b) with the extragranular excipients;
(d) adding at least one lubricant to the blended composition of step (c);
(e) compressing the blended composition of step (d) to obtain a tablet composition; and
(f) optionally coating the tablet composition.

In a tenth aspect, the present invention relates to the pharmaceutical composition of the first or third aspect or the pharmaceutical batch of the sixth aspect obtained by the process of the ninth aspect.

In an eleventh aspect, the present invention relates to a cardboard box with a patient information leaflet comprising at least one aluminium/aluminium or aluminium/PVC blister pack of at least 4 units of the pharmaceutical composition of the first or third aspect.

In a twelfth aspect, the present invention relates to a cardboard box with patient information leaflet comprising at least an HDPE bottle with at least 4 units of the pharmaceutical composition of the first or third aspect.

In a thirteenth aspect, the present invention relates to a pharmaceutical composition comprising a combination of 400 mg of sofosbuvir and 90 mg of ledipasvir or a pharmaceutically acceptable salt or hydrate thereof.

In a fourteenth aspect, the present invention relates to the pharmaceutical composition of the thirteenth aspect for use in the treatment of chronic hepatitis C.

### DETAILED DESCRIPTION OF THE INVENTION

The present invention provides a highly stable pharmaceutical compositions of sofosbuvir or a pharmaceutically acceptable salt thereof in the form of immediate release tablets. The pharmaceutical composition of the present invention increase the concentration of sofosbuvir per total weight of the tablet, which permits smaller tablets to be used to deliver the same dosage of the drug. The stable immediate release tablets as disclosed herein can be stored at temperatures higher than 25° C. and are stable even when packaged in aluminium/PVC. Another advantage of the compositions and processes as herein disclosed is that small tablets which are easy to swallow are provided.

The term “stable” as used herein refers to a pharmaceutical composition comprising sofosbuvir wherein the total content of impurities originating from the decomposition of sofosbuvir does not exceed 5% area, preferably 3% area, more preferably 2% area and most preferably 1% area determined by liquid chromatography (HPLC) at 210 nm if such a composition is stored for 2 months at 40° C. and 75% relative humidity (RH).

The term “active ingredient” or “active agent” refers to a therapeutically active compound, as well as any prodrugs thereof and pharmaceutically acceptable salts, hydrates and solvates of the compound and the prodrugs.

An immediate release tablet as herein disclosed has to be understood as a tablet having a dissolution performance such as 60% or more of the active agent contained in said pharmaceutical composition dissolves within 60 minutes (min). In a preferred embodiment, the immediate release composition as herein disclosed releases at least 80% of the active agent in 60 min. In another embodiment, the pharmaceutical composition as herein disclosed releases at least 80% of the active agent in 45 min, preferably in 35 min and more preferably in 30 min. In another embodiment, the pharmaceutical composition as herein disclosed releases at least 80% of the active agent in 30 min and at least a 95% of the active agent in 60 min. In a most preferred embodiment, the pharmaceutical composition as herein disclosed releases at least 80% of the active agent in 15 min. and at least 95% of the active agent in 60 min. The dissolution test for an immediate release pharmaceutical composition comprising the active agent as herein disclosed is performed in the following conditions: USP Apparatus: II (Paddles). Speed: 75 rpm. Medium: phosphate buffer pH 6.8. Wavelength 210 nm.

In a further embodiment of the pharmaceutical composition of the first aspect, the pharmaceutical composition comprises from more than about 35% to about 50% w/w of sofosbuvir as a free base, preferably comprises from about 36% to about 40% w/w of sofosbuvir as a free base, more preferably comprises from 36% to 38% w/w of sofosbuvir as a free base in respect of the total amount of the pharmaceutical composition.

The term “% w/w of sofosbuvir as a free base in respect of the total amount of the pharmaceutical composition” as used herein, refers to the amount of sofosbuvir or the pharmaceutically acceptable salt thereof, in the pharmaceutical composition or in the granulate, being calculated considering the equivalent amount of sofosbuvir as free base.

Sofosbuvir can be crystalline or amorphous. Examples of preparing crystalline and amorphous forms of sofosbuvir are disclosed in US 2010/0298257 and US 2011/0251152. Polymorphic forms 1-6 of sofosbuvir disclosed in US 2010/0298257 and/or US 2011/0251152 have the following characteristic X-ray powder diffraction (XRPD) pattern 2θ-values measured according to the XRPD methods disclosed herein:

(1) 2θ-reflections (°) at about: 5.2, 7.5, 9.6, 16.7, 18.3, and 22.2 (Form 1);
(2) 2θ-reflections (°) at about: 5.0, 7.3, 9.4, and 18.1 (Form 1);
(3) 2θ-reflections (°) at about: 4.9, 6.9, 9.8, 19.8, 20.6, 24.7, and 26.1 (Form 2);
(4) 2θ-reflections (°) at about: 6.9, 9.8, 19.7, 20.6, and 24.6 (Form 3);
(5) 2θ-reflections (°) at about: 5.0, 6.8, 19.9, 20.6, 20.9, and 24.9 (Form 4);
(6) 2θ-reflections (°) at about: 5.2, 6.6, 7.1, 15.7, 19.1, and 25.0 (Form 5); and
(7) 2θ-reflections (°) at about: 6.1, 8.2, 10.4, 12.7, 17.2, 17.7, 18.0, 18.8, 19.4, 19.8, 20.1, 20.8, 21.8, and 23.3 (Form 6).

The pharmaceutical compositions of the prior-art use different crystalline forms of sofosbuvir to develop a dosage form to be administered in human. None of them describe the use of the amorphous form of sofosbuvir in an oral pharmaceutical composition. The present invention provides a highly stable pharmaceutical compositions of the amorphous form of sofosbuvir in the form of immediate release tablets, which possess advantageous stable dissolution profiles, disintegrates rapidly in aqueous solutions and has a low content of total impurities.

In a further embodiment of the pharmaceutical composition as herein disclosed, the pharmaceutical composition comprises the amorphous form of sofosbuvir. Preferably, the sofosbuvir has a XRPD pattern as depicted in FIG. 1. XRPD was performed as explained in the examples.

In a further embodiment of the pharmaceutical composition as herein disclosed, the pharmaceutical composition comprises a crystalline form of sofosbuvir. Preferably, the pharmaceutical composition comprises crystalline form 1 of sofosbuvir characterised by at least X-ray powder diffraction peaks 5.2, 7.5, 9.6, and 18.3° 2θ (±0.2° 2θ). Preferably, the pharmaceutical composition consists essentially of crystalline form 1 of sofosbuvir characterised by at least X-ray powder diffraction peaks 5.2, 7.5, 9.6, and 18.3° 2θ (±0.2° 2θ).

In a further embodiment of the pharmaceutical composition as herein disclosed, the pharmaceutical composition comprises crystalline form 6 of sofosbuvir characterised by at least X-ray powder diffraction peaks 6.1, 8.2, 10.4, and 12.7° 2θ) (±0.2° 2θ). Preferably, the pharmaceutical composition consists essentially of crystalline form 6 of sofosbuvir characterised by at least X-ray powder diffraction peaks 6.1, 8.2, 10.4, and 12.7° 2θ (±0.2° 2θ).

When the pharmaceutical composition as herein disclosed comprises excipients such as a filler, a lubricant, a glidant or a disintegrant, these excipients are pharmaceutically acceptable. The term “pharmaceutically acceptable” indicates that the substance or composition must be compatible chemically and/or toxicologically, with the other ingredients comprising a formulation, the mammal being treated therewith, and/or the route of administration of the composition.

In a further embodiment of the pharmaceutical composition of the first aspect, the pharmaceutical composition comprises a granular component comprising sofosbuvir or a pharmaceutically acceptable salt thereof, and at least one filler. Preferably the total amount of filler or fillers present in the granular component ranges from 30 to 64% w/w, more preferably ranges from 35 to 62% w/w in respect of the total amount of the pharmaceutical composition.

The term “granular component” or “granulate” as used herein refers to the part of the pharmaceutical composition that has been obtained by granulating a powder into larger particles herein called granules or granulate. Said granulation can be either wet granulation or dry granulation.

The term “filler” as used herein refers to pharmaceutically acceptable excipients which are added to the bulk volume of the active agent making up the solid composition. As a result, the size of the solid composition increases, which makes its size suitable for handling. Fillers are convenient when the dose of drug per solid composition is low and the solid composition would otherwise be too small. In a preferred embodiment of the pharmaceutical composition as herein disclosed, said pharmaceutical composition comprises at least a filler. Preferably, the total amount of filler or fillers present in the pharmaceutical composition ranges from 30 to 64% w/w in respect of the total amount of the pharmaceutical composition. More preferably, the total amount of acid or acids present in the pharmaceutical composition ranges from 35 to 62% w/w in respect of the total amount of the pharmaceutical composition. Even more preferably, the total amount of filler or fillers present in the pharmaceutical composition ranges from 45 to 60% w/w in respect of the total amount of the pharmaceutical composition.

In a preferred embodiment of the pharmaceutical composition as herein disclosed, said pharmaceutical composition further comprises a filler selected from the group consisting of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil (type I), inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, and mixtures thereof. Preferably, the pharmaceutical composition further comprises a filler selected from the group consisting of dicalcium phosphate, cellulose, compressible sugars, dibasic calcium phosphate dihydrate, lactose, mannitol, microcrystalline cellulose, starch, tribasic calcium phosphate, and mixtures thereof, more preferably the pharmaceutical composition comprises microcrystalline cellulose, mannitol or mixtures thereof.

As used herein, “lubricant” means a substance that reduces friction between the composition of the present invention and the surfaces of the apparatus used to compact the composition into a compressed form. In a preferred embodiment of the pharmaceutical composition as herein disclosed, said pharmaceutical composition comprises at least a lubricant. Preferably, the total amount of lubricant or lubricants present in the pharmaceutical composition ranges from 0.5 to 5% w/w in respect of the total amount of the pharmaceutical composition. More preferably, the total amount of lubricant or lubricants present in the pharmaceutical composition ranges from 1 to 3% w/w in respect of the total amount of the pharmaceutical composition.

In a preferred embodiment of the pharmaceutical composition as herein disclosed, said pharmaceutical composition comprises at least a lubricant selected from calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate, and mixtures thereof. Preferably, the pharmaceutical composition comprises at least a lubricant selected from calcium stearate, magnesium stearate, polyethylene glycol, sodium stearyl fumarate, stearic acid, talc, or mixtures thereof, more preferably the pharmaceutical composition comprises magnesium stearate.

As used herein, “glidant” means a substance which improves the flow characteristics of powder mixtures in the dry state. In a preferred embodiment of the pharmaceutical composition as herein disclosed, said pharmaceutical composition comprises at least a glidant. Preferably, the total amount of glidant or glidants present in the pharmaceutical composition ranges from 0.01 to 3% w/w in respect of the total amount of the pharmaceutical composition. More preferably, the total amount of glidant or glidants present in the pharmaceutical composition ranges from 0.05 to 1.5% w/w in respect of the total amount of the pharmaceutical composition.

In a preferred embodiment of the pharmaceutical composition as herein disclosed, said pharmaceutical composition comprises at least a glidant selected from the group consisting of colloidal silicon dioxide, talc, starch, starch derivatives, and mixtures thereof. Preferably, the pharmaceutical composition comprises colloidal silicon dioxide.

As used herein, “disintegrant” means a substance or a mixture of substances added to a tablet to facilitate its breakup or disintegration after administration. In a preferred embodiment of the pharmaceutical composition as herein disclosed, said pharmaceutical composition comprises at least a disintegrant. Preferably, the total amount of disintegrant or disintegrants present in the pharmaceutical composition ranges from 1 to 15% w/w in respect of the total amount of the pharmaceutical composition. More preferably, the total amount of disintegrant or disintegrants present in the pharmaceutical composition ranges from 2 to 10% w/w in respect of the total amount of the pharmaceutical composition.

In a preferred embodiment of the pharmaceutical composition as herein disclosed, said pharmaceutical composition comprises at least a disintegrant selected from water-soluble disintegrants, such as starch, pregelatinized starch, sodium carboxymethyl cellulose, sodium starch glycolate, povidone, croscarmellose sodium, crospovidone, microcrystalline cellulose, low-substituted hydroxypropyl cellulose and mixtures thereof. Preferably, the pharmaceutical composition comprises at least a disintegrant selected from starch, pregelatinized starch, sodium starch glycolate, povidone, croscarmellose sodium, crospovidone, microcrystalline cellulose, and mixtures thereof, more preferably the pharmaceutical composition comprises croscarmellose sodium, crospovidone or mixtures thereof.

In a further embodiment of the pharmaceutical composition as herein disclosed, the pharmaceutical composition comprises between 100 and 1000 mg of sofosbuvir per tablet, preferably comprises about 400 mg of sofosbuvir per tablet.

Tablets may be prepared according to methods known in the art, including dry granulation (e.g., roller compaction), wet granulation (e.g., fluid bed granulation and high shear granulation), and direct compression, and the type of excipients used will vary accordingly. It has been found that dry granulation is particularly suitable for providing high strength, low breakage tablets comprising relatively high concentrations of crystalline sofosbuvir and also amorphous form of sofosbuvir (e.g., about 36-37%), on a scale suitable for commercial production. Suitable dry granulated tablets comprise granules comprising sofosbuvir and one or more of a filler, a disintegrant, a glidant, and a lubricant, wherein the granules are mixed with one or more of a filler, a disintegrant, a glidant, and a lubricant to form a blended composition that is compressed to form tablets.

In a further embodiment of the pharmaceutical composition as herein disclosed, the pharmaceutical compositions are manufactured by direct compression, dry granulation or wet granulation, preferably the pharmaceutical compositions are manufactured by direct compression or dry granulation. More preferably the pharmaceutical composition is in the form of a compressed tablet or granules or capsule.

In a further embodiment of the pharmaceutical composition as herein disclosed, the tablets are packaged in a blister pack of aluminium/PVC or aluminium/aluminium. In a further embodiment of the granulate of the second aspect, the granulate comprises from about 30% to about 50% w/w of sofosbuvir as free base, preferably comprises from 35% to 45% w/w of sofosbuvir as free base in respect of the total amount of the granulate.

The granulates as herein disclosed and the powders as herein disclosed, i.e. the mixture formed with granulate and the extra-granular ingredients, have a good flowability. Good flow characteristics are necessary because the mechanical action of the tablet press requires a volume of fill.

In a further embodiment of the granulate as herein disclosed, the total amount of disintegrant or disintegrants present in the granulate ranges from 0.2 to 5% w/w, preferably ranges from 0.5 to 4% w/w, more preferably ranges from 1 to 3% w/w.

In a further embodiment of the granulate as herein disclosed, the total amount of filler or fillers present in the granulate ranges from 35% to 70% w/w, preferably ranges from 40 to 65% w/w, more preferably ranges from 45 to 60% w/w.

In a further embodiment of the granulate as herein disclosed, the granulate further comprises at least one glidant and at least one lubricant. Preferably the total amount of glidant or glidants present in the granulate ranges from 0.05% to 3% w/w, more preferably ranges from 0.1 to 1.5% w/w. In a further embodiment of the granulate as herein disclosed, the total amount of lubricant or lubricants present in the granulate ranges from 0.05% to 3% w/w, preferably ranges from 0.1 to 1.5% w/w.

In a further embodiment of the pharmaceutical batch of the sixth aspect, the content of sofosbuvir or the pharmaceutically acceptable salt thereof is uniform. Preferably, the pharmaceutical batch comprises at least 50,000 units, more preferably, the pharmaceutical batch comprises at least 100,000 units.

In a further embodiment of the pharmaceutical batch as herein disclosed, the tablets are packaged in a blister pack of aluminium/PVC or aluminium/aluminium or in a high-density polyethylene (HDPE) bottle. Preferably, the HDPE bottle as herein disclosed, has a polypropylene child-resistant closure with induction-seal liners.

The term “batch” as used herein refers to a specific quantity of a drug or other material that is intended to have uniform character and quality, within specified limits, and is produced according to a single manufacturing order during the same cycle of manufacture. A batch, in the case of a drug product produced by continuous process, is a specific identified amount produced in a unit of time or quantity in a manner that assures its having uniform character and quality within specified limits (Code of Federal Regulations Title 21, Food and Drug Administration department of Health and Human Services, Subchapter C, Section 210.3 (b) (2) and (10)).

The term “pharmaceutical batch” as used herein refers to a batch as defined above of a pharmaceutical composition manufactured in accordance with the principles and guidelines of Good Manufacturing Practice (GMP) at an industrial scale and which is intended for commercialization (Directive 91/356/EEC).

The pharmaceutical composition may be manufactured at laboratory scale, not necessarily following GMP and not intended for commercialization. The pharmaceutical composition may also be manufactured for validation, following GMP. A batch of a pharmaceutical composition which is manufactured for validation is called “pilot batch”.

Each pharmaceutical batch of finished product must fulfil the regulatory requirements of the corresponding Medicine Agency before being released for sale or supply, such as impurities thresholds and stability data.

The term “uniform” as used herein refers to the content of the active ingredient in the tablets of a pharmaceutical batch has to be homogeneous. According to the FDA criteria, uniformity is considered as achieving 90-110% potency of the theoretical strength with a relative standard deviation (RSD) of less than 5% for all samples (Guidance for Industry ANDA's: Blend Uniformity Analysis, published August 1999).

For the release of a pharmaceutical batch the distribution of the active ingredient in the tablets has to be homogeneous, that is, content uniformity is required. All batches are expected to be uniform within normal process variation. Process validation studies are conducted prior to the marketing of drug product to assure that production processes are controlled. The test batch is manufactured prior to validation, yet it is the basis on which an application is approved (MANUAL OF POLICIES AND PROCEDURES, MAPP 5225.1).

It is essential, therefore, to assure that the test batch is uniform. In-process tests for uniformity should be conducted throughout the entire production process, e.g., at commencement or completion of significant phases (21 CFR 211.110). These tests should be designed to detect potential in-process anomalies (MAPP 5225.1).

The eighth aspect of present invention relates to a drug product, comprising at least i) a pharmaceutical composition in the form of a tablet comprising sofosbuvir or a pharmaceutically acceptable salt thereof, and ii) a blister pack packaging the pharmaceutical composition (i).

The term “drug product” refers to the dosage form in the final primary packaging intended for marketing, i.e. the drug product is the final market product.

In a further embodiment of the drug product of the eighth aspect, the drug product comprises the pharmaceutical composition in the form of an oral tablet as herein disclosed. Preferably, the blister pack of the drug product is an aluminium/PVC blister or an aluminium/aluminium blister.

The term “blister” or bubble pack refers to a sheet in a package construction with recesses designed to hold dosage forms. The sheet may be a plastic, a foil, or combination thereof. Normally, a blister is a product consisting of a flat structure in which blisters are formed, generally by means of a heating process, into which the single elements to be packaged are inserted. The blisters are then hermetically sealed using flat strips of appropriate thermomoldable materials (plastics, aluminium, paper), which represent the frangible element through which it is then possible to remove the product. The primary component of a blister pack is a cavity or pocket made from a formable web, usually a thermoformed plastic. This usually has a backing of paperboard or a lidding seal of aluminum foil or plastic. An aluminium/PVC blister refers to a blister the thermomoldable material is from PVC and the backing is a lidding seal of aluminium foil. Blister packs are commonly used as unit-dose packaging for pharmaceutical tablets, capsules or lozenges. Blister packs can provide barrier protection for shelf life requirements, and a degree of tamper resistance. A series of blister cavities is sometimes called a blister card or blister strip as well as blister pack. The difference between a strip pack and blister pack is that a strip pack does not have thermo-formed or cold formed cavities; the strip pack is formed around the tablet at a time when it is dropped to the sealing area between sealing moulds. In some parts of the world the pharmaceutical blister pack is known as a Push-Through-Pack (PTP), an accurate description of two key properties (i) the lidding foil is brittle allowing to press the product out while breaking the lidding foil and (ii) a semi-rigid formed cavity being sufficiently collapsable to be able to dispense the tablet or capsule by means of pressing it out with your thumb.

The main advantages of unit-dose blister packs over other methods of packing pharmaceutical products are the assurance of product/packaging integrity (including shelf life) of each individual dose and the possibility to create a compliance pack or calendar pack by printing the days of the week above each dose. Blister packs can be created by means of a form-fill-seal process at the pharmaceutical company or designated contract packer. A form-fill-seal process means that the blister pack is created from rolls of flat sheet or film, filled with the pharmaceutical product and closed (sealed) on the same equipment. Such equipment is called a blisterline. There are two types of blister machine's design: rotary and flat-plate.

Furthermore, the benefits of blister pack vs. traditional bottles are clear and cross multiple market segments. In terms of safety, studies have consistently shown that blister packaging outperforms child-resistant bottles. The child-resistant features used in bottles often become disabled. Package reclosing is not necessary to maintain child-resistance. Unlike many child-resistant bottle solutions, blister pack does not require additional labels, cartons or plastic components to become child-resistant.

Studies also show the direct connection between well-designed unit dose packaging and improved patient compliance/adherence. Blister pack helps to achieve several goals:
More accurate dispensing: Pre-packaged blister pack cards reduce bulk dispensing and handling errors.
More accurate dosing: Unit-dose blister pack cards are easier to use, particularly for patients taking multiple pills per dose and those who have difficulty remembering proper dosage protocols.
Push-Through-Pull-Tab Indicator reduces the chance of patient dosing errors.
Improved refill rates: Blister pack makes it easier for patients to manage their own supply of products. With bottles, many patients don't realize they need to refill their prescription until they are down to the last pill. This can lead to missed doses until such a time the patients can get the refilled prescription. With the calendar format of blister pack you can print refill reminder. For example, you may choose to have the package indicate it is “time to refill” when there are 5 doses remaining on the card.
Ease of use: In addition, 100% of seniors were able to successfully access their medications and comply with dosing requirements with senior-friendly blister packaging. This not only improves patient outcome; it can help to increase overall product sales.

In a further embodiment of the process of the ninth aspect, the intragranular composition comprises sofosbuvir or a pharmaceutically acceptable salt thereof, a first intragranular filler, optionally a second intragranular filler, an intragranular disintegrant, optionally an intragranular glidant, and optionally an intragranular lubricant; and the extragranular composition comprises a first extragranular filler, optionally a second extragranular filler, optionally an extragranular glidant, optionally an extragranular disintegrant, and an extragranular lubricant.

The term “extragranular component” or “extragranular composition” as used herein refers to the set of ingredients in the pharmaceutical composition which have not been granulated and which do not form part of the granular component.

In a further embodiment of the pharmaceutical composition of the thirteenth aspect, the pharmaceutical composition comprises from about 25% to about 50% w/w of sofosbuvir as free base and from about 5% to about 18% of ledipasvir as free base in respect of the total amount of the pharmaceutical composition. Preferably, the composition comprises from about 30% to about 40% w/w of sofosbuvir as free base and from about 8% to about 14% of ledipasvir as free base in respect of the total amount of the pharmaceutical composition.

Another aspect of the present invention relates to the combination of sofosbuvir and ledipasvir which has been found to be effective for the treatment of chronic hepatitis C genotype 1 infection in adults. The pharmaceutical composition comprising the combination of both active ingredients is stable and has a fast onset.

In a further embodiment of the pharmaceutical composition of the thirteenth aspect, the pharmaceutical composition comprises a pharmaceutical composition as defined in the patent application WO2013082003 (e.g. table 1A, table 2 or table 3) or the pharmaceutical composition of the first or third aspect.

As used herein, the term “unit dose” or “unit dosage” or “unit” refers to a physically discrete unit that contains a predetermined quantity of active ingredient calculated to produce a desired therapeutic effect. The unit dose or unit dosage or unit may be in the form of a tablet, capsule, sachet, etc. referred to herein as a “unit dosage form”.

All percentages, parts, and ratios herein are w/w unless specifically noted otherwise. As used herein, the term “about” refers preferably to a range that is ±10%, preferably ±5%, or more preferably ±1% of a value with which the term is associated.

Unless otherwise indicated, all the analysis methods are carried out according to the European Pharmacopoeia 7th edition.

### DESCRIPTION OF THE FIGURES

FIG. 1: X-ray powder diffraction pattern (XRD) of sofosbuvir amorphous form.

### EXAMPLES

### Examples 1

### Sofosbuvir Tablet Composition

| Intragranular | |
|---|---|
| Sofosbuvir (amorphous) (mg) | 400.00 |
| Colloidal silicon dioxide (mg) | 5.40 |
| Crospovidone (mg) | 25.00 |
| Magnesium stearate (mg) | 9.00 |
| Mannitol (mg) | 360.00 |
| Microcrystalline cellulose (mg) | 220.00 |
| Extragranular | |
| Colloidal silicon dioxide (mg) | 0.60 |
| Crospovidone (mg) | 25.00 |
| Magnesium stearate (mg) | 0.83 |
| Microcrystalline cellulose (mg) | 30.00 |
| Total (mg/tablet) | 1084.00 |

### Examples 2

### Sofosbuvir Tablet Composition

| Intragranular | |
|---|---|
| Sofosbuvir (form I) (mg) | 400.00 |
| Colloidal silicon dioxide (mg) | 5.40 |
| Croscarmellose sodium (mg) | 25.00 |
| Magnesium stearate (mg) | 9.00 |
| Mannitol (mg) | 360.00 |
| Extragranular | |
| Colloidal silicon dioxide (mg) | 0.60 |
| Croscarmellose sodium (mg) | 25.00 |
| Magnesium stearate (mg) | 9.00 |
| Microcrystalline cellulose (mg) | 250.00 |
| Total (mg/tablet) | 1084.00 |

### Examples 3

### Sofosbuvir Tablet Composition

| Intragranular | |
|---|---|
| Sofosbuvir (form VI) (mg) | 400.00 |
| Colloidal silicon dioxide (mg) | 5.40 |
| Croscarmellose sodium (mg) | 25.00 |
| Magnesium stearate (mg) | 9.00 |
| Mannitol (mg) | 360.00 |
| Microcrystalline cellulose (mg) | 220.00 |
| Extragranular | |
| Colloidal silicon dioxide (mg) | 0.60 |
| Croscarmellose sodium (mg) | 25.00 |
| Magnesium stearate (mg) | 9.00 |
| Microcrystalline cellulose (mg) | 30.00 |
| Total (mg/tablet) | 1084.00 |

### Example 4

### Manufacture of Sofosbuvir Tablets of Examples 1, 2 and 3 by Dry Granulation

(1) A composition comprising sofosbuvir and the intragranular excipients (mannitol, crospovidone or croscarmellose sodium, colloidal silicon dioxide and optionally microcrystalline cellulose) was sifted through a 0.85 mm screen and added to a blender and blended for about 10-15 minutes at 34 rpm to obtain an initial blend. Separately, the intragranular magnesium stearate was passed through a 0.85 mm screen and mixed with the initial blend, and blended for about 5 minutes at 34 rpm to obtain an intragranular blend.

(2) Separately, the extragranular excipients microcrystalline cellulose, crospovidone or croscarmellose sodium, and colloidal silicon dioxide were sifted through a 0.85 mm screen for use in the final blending (step (4), below).

(3) The intragranular blend comprising sofosbuvir, mannitol, crospovidone or croscarmellose sodium, colloidal silicon dioxide, magnesium stearate and optionally microcrystalline cellulose was passed through a roller compactor equipped until granulation was achieved. Ribbons were produced using flat straight-grooved rollers. Upon passing through the roller compactor, the granules were screened through a 1.4 mm screen. The milled/sifted granule material was passed to the final blending step.

(4) The milled/sifted granules from step (3) and the sifted extragranular excipients (microcrystalline cellulose, mannitol, croscarmellose sodium and silicon dioxide) from step (2) were added to a blender and blended for about 15 minutes at 34 rpm. Separately, magnesium stearate was passed through a 0.85 mm screen. The magnesium stearate was added to the blender and blended for about 5 minutes at 34 rpm to obtain a final powder blend comprising 37% w/w sofosbuvir. Blend uniformity samples were taken prior to removing the blend from the blender.

(5) The final blend was compressed into tablets using a tablet press to obtain 1084 mg uncoated tablets comprising about 400 mg of sofosbuvir.

### Example 5

### Stability

Tablets of example 1 were packaged in blisters packs of aluminium/PVC. Tablets of example 1 were subjected to accelerated stability tests at times 0 and 72 hours, at 40° C. and 75% Relative Humidity (RH) and 50° C. and saturated RH.

TABLE A
Accelerated stability tests (72 hours).
Percentage of total impurities.

| Batch | t = 0 | 40° C./75% RH | 50° C./sat RH |
|---|---|---|---|
| 5 | 0.13 | 0.17 | 0.19 |
| 6 | 0.11 | 0.12 | 0.26 |

### Example 6

### X-Ray Powder Diffraction

X-Ray Powder Diffraction was performed in a Bruker D8 Advance A25 diffractometer with a θ:2θ configuration and Bragg-Brentano geometry with a copper anode tube and the detection was performed in Lynxeye Bruker (linear detector).

The diffractogram is obtained for 2θ angles ranging from 3° to 70° with a step of 0.03° each second. The tube set-up is 40 kV and 30 mA, incident-beam divergence-limiting slit 12 mm, static sample, diffracted-beam receiving slit 0.2 mm and Nickel filter. The sample was stirred at 30 rpm during the test.

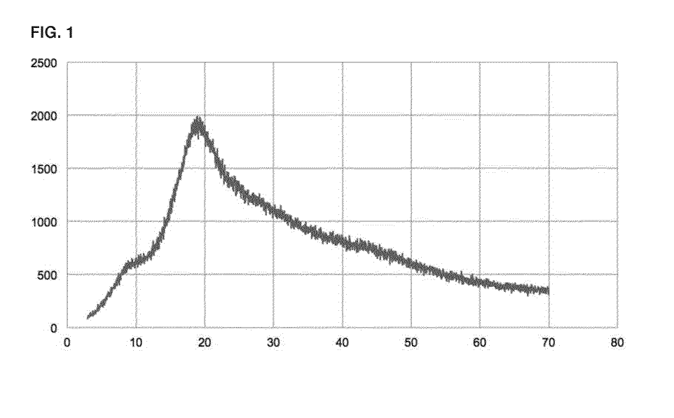

X-ray powder diffraction pattern (XRPD) of sofosbuvir amorphous form is depicted in FIG. 1.

1. An oral solid pharmaceutical composition comprising sofosbuvir or a pharmaceutically acceptable salt thereof, wherein said pharmaceutical composition comprises:
a) more than about 35% w/w of sofosbuvir as a free base in respect of the total amount of the pharmaceutical composition; and
b) at least one pharmaceutically acceptable excipient.

2.-46. (canceled)

47. The pharmaceutical composition according to claim 1, wherein the pharmaceutical composition comprises the amorphous form of sofosbuvir.

48. The pharmaceutical composition according to claim 1, wherein the pharmaceutical composition comprises a crystalline form of sofosbuvir.

49. The pharmaceutical composition according to claim 1, wherein the pharmaceutical composition consists essentially of crystalline form 1 of sofosbuvir, characterised by at least X-ray powder diffraction peaks 5.2, 7.5, 9.6, and 18.3° 2θ (±0.2° 2θ) or wherein the pharmaceutical composition consists essentially of crystalline form 6 of sofosbuvir, characterised by at least X-ray powder diffraction peaks 6.1, 8.2, 10.4, and 12.7° 2θ (±0.2° 2θ).

50. The pharmaceutical composition according to claim 1, wherein the pharmaceutical composition comprises a granular component comprising sofosbuvir or a pharmaceutically acceptable salt thereof, and at least one filler.

51. The pharmaceutical composition according to claim 1, wherein the pharmaceutical composition comprises a granular component comprising sofosbuvir or a pharmaceutically acceptable salt thereof, and at least one filler, wherein the total amount of filler or fillers present in the pharmaceutical composition ranges from 30 to 64% w/w in respect of the total amount of the pharmaceutical composition, and/or the pharmaceutical composition comprises microcrystalline cellulose, mannitol or mixtures thereof.

52. The pharmaceutical composition according to claim 1, wherein the pharmaceutical composition comprises at least a lubricant.

53. The pharmaceutical composition according to claim 1, wherein the pharmaceutical composition comprises at least a lubricant, wherein the total amount of lubricant or lubricants present in the pharmaceutical composition ranges from 0.5 to 5% w/w in respect of the total amount of the pharmaceutical composition, and/or the pharmaceutical composition comprises magnesium stearate.

54. The pharmaceutical composition according to claim 1, wherein the pharmaceutical composition comprises at least a disintegrant.

55. The pharmaceutical composition according to claim 1, wherein the pharmaceutical composition comprises at least a disintegrant, wherein the total amount of disintegrant or disintegrants present in the pharmaceutical composition ranges from 1 to 15% w/w in respect of the total amount of the pharmaceutical composition, and/or the pharmaceutical composition comprises croscarmellose sodium, crospovidone or mixtures thereof.

56. The pharmaceutical composition according to claim 1, wherein the pharmaceutical composition comprises at least a glidant.

57. The pharmaceutical composition according to claim 1, wherein the pharmaceutical composition comprises at least a glidant, wherein the total amount of glidant or glidants present in the pharmaceutical composition ranges from 0.01 to 3% w/w in respect of the total amount of the pharmaceutical composition, and/or the pharmaceutical composition comprises colloidal silicon dioxide.

58. The pharmaceutical composition according to claim 1, wherein the pharmaceutical composition comprises between 100 and 1,000 mg of sofosbuvir per tablet or comprises about 400 mg of sofosbuvir per tablet.

59. The pharmaceutical composition according to claim 1, wherein the pharmaceutical composition is manufactured by direct compression, dry granulation or wet granulation, and/or the pharmaceutical composition is in the form of a compressed tablet or granules or capsule.

60. The pharmaceutical composition according to claim 1, wherein the pharmaceutical composition is manufactured by direct compression or dry granulation.

61. The pharmaceutical composition according to claim 1, wherein the pharmaceutical composition comprises a granulate comprising sofosbuvir or a pharmaceutically acceptable salt thereof, wherein the granulate comprises from about 30% to about 50% w/w of sofosbuvir as free base in respect of the total amount of the granulate and at least one disintegrant and at least one filler.

62. The pharmaceutical composition according to claim 1, wherein the pharmaceutical composition is in the form of an oral tablet packaged in a blister pack of aluminium/PVC or aluminium/aluminium or in a high density polyethylene (HDPE) bottle.

63. The pharmaceutical composition according to claim 1, wherein the pharmaceutical composition comprises a combination of 400 mg of sofosbuvir and 90 mg of ledipasvir or a pharmaceutically acceptable salt or hydrate thereof.

64. A method of treating hepatitis C comprising the administration of the pharmaceutical composition according to claim 1.

65. A process for the manufacture of the pharmaceutical composition as defined in claim 1, wherein the process comprises the following steps:
(a) providing an intragranular mixture of sofosbuvir or a pharmaceutically acceptable salt thereof and at least one disintegrant and at least one filler;
(b) granulating the mixture by a dry granulation process to produce a granulate;
(c) blending the granulate of step (b) and the extragranular excipients;
(d) adding at least one lubricant to the blended composition of step (c);
(e) compressing the blended composition of step (d) to obtain a tablet composition; and
(f) optionally coating the tablet composition.
